(19) United States
(12) Patent Application Publication
(10) Pub. No.: US 2016/0317615 A1
(43) Pub. Date: Nov. 3, 2016

(54) T CELL MODULATOR

(71) Applicants: Jose Javier Lopez GONZALEZ, Chula Vista, CA (US); Thomas ICHIM, San Diego, CA (US)

(72) Inventors: Jose Javier Lopez GONZALEZ, Chula Vista, CA (US); Thomas ICHIM, San Diego, CA (US)

(21) Appl. No.: 15/144,680

(22) Filed: May 2, 2016

Related U.S. Application Data
(60) Provisional application No. 62/156,122, filed on May 1, 2015.

(51) Int. Cl. A61K 38/17; A61K 35/15; A61K 38/10

(52) U.S. Cl. CPC A61K 38/1709; A61K 38/10; A61K 35/15

(57) ABSTRACT

Compositions, methods of use, and pharmaceutical preparations useful for modulation of immune responses are disclosed herein. In one embodiment a composition is extracted from immune organs or leukocytes derived from members of the chondrichthyes family through dialysis. Said immune modulator is useful for treatment of conditions requiring stimulations or regulation of immunity. In one embodiment, said immunomodulator controls T cell activation by modulation of cytokine production.

### CROSS REFERENCE TO RELATED APPLICATIONS

This application is related to, and claims the benefit of, U.S. Provisional Application No. 62/156,122, filed May 1, 2015. The above-identified priority patent application is incorporated herein by reference in its entirety.

### FIELD OF INVENTION

The present technology relates to compositions, methods of use, and pharmaceutical preparations useful for modulation of immune responses. In one embodiment a composition is extracted from immune organs or leukocytes derived from members of the chondrichthyes family through dialysis. Said immune modulator is useful for treatment of conditions requiring stimulations or regulation of immunity. In one embodiment, said immunomodulator controls T cell activation by modulation of cytokine production.

### BACKGROUND OF THE INVENTION

Cancer has historically been treated with surgery, radiation, chemotherapy, and hormone therapy. More recently, advances in understanding of the immune system's role in cancer have led to immunotherapy becoming an important treatment approach. Cancer immunotherapy began with treatments that nonspecifically activated the immune system and had limited efficacy and/or significant toxicity. In contrast, new immunotherapy treatments can activate specific, important immune cells, leading to improved targeting of cancer cells, efficacy, and safety. Within the immunotherapy category, treatments have included cytokine therapies, antibody therapies, and adoptive cell therapies.

In 1986, interferon-alpha became the first cytokine approved for cancer patients. In 1992, interleukin-2, or IL-2, was the second approved cytokine in cancer treatment, showing efficacy in melanoma and renal cell cancer. IL-2 does not kill cancer cells directly, but instead nonspecifically activates and stimulates the growth of the body's own T cells which then combat the tumor. Although interferon-a, IL-2, and subsequent cytokine therapies represent important advances in cancer treatment, they are generally limited by toxicity and can only be used in a limited number of cancers and patients.

After cytokines set the stage for immunotherapy, antibody therapies represented the next significant advance, with targeted specificity and a generally better-tolerated side effect profile. Monoclonal antibodies, or mAbs, are designed to attach to proteins on cancer cells, and once attached, the mAbs can make cancer cells more visible to the immune system, block growth signals of cancer cells, stop new blood vessels from forming, or deliver radiation or chemotherapy to cancer cells. The first FDA approved mAb specifically for cancer was Rituxan in 1997, and since then, many other antibodies have received approval, including Herceptin, Avastin, Campath, Erbitux, and Vectibix. More recently, antibodies have been conjugated with cytotoxic drugs to increase activity. The first approved antibody drug conjugate was Mylotarg in 2000, followed by Adcetris in 2011 and Kadcycla in 2013.

The next important advance has been the development of antibodies that target T cell checkpoint pathways, which are means by which cancer cells are able to inhibit or turn down the body's immune response to cancer. These treatments have shown an ability to activate T cells, shrink tumors, and improve patient survival. In 2011, Yervoy became the first checkpoint inhibitor approved by the FDA. Recent clinical data from checkpoint inhibitors such as nivolumab and Keytruda have confirmed both the approach and the importance of T cells as promising tools for the treatment of cancer. Despite these many advances, a significant unmet need in cancer still persists.

The invention provides for production of T cell modulator (TCM), a leukocyte extract capable of upregulating or downregulating immune responses based on physiological need of the host. In one aspect the invention teaches isolation of a leukocyte dialysate possessing a molecular weight of approximately 12 kDa or less.

### BRIEF DESCRIPTION OF THE FIGURES

FIG. 1A is a bar graph showing percentage of viability of HeLa compared to Control.

FIG. 1B is a bar graph showing percentage of viability of PC-3 compared to Control.

FIG. 1C is a bar graph showing percentage of viability of DU-145 compared to Control.

FIG. 1D is a bar graph showing percentage of viability of 3T3 compared to Control.

FIG. 2A is a bar graph showing IFN-Gamma Con A Stimulation.

FIG. 2B is a bar graph showing IL-4 Con A Stimulation.

FIG. 2C is a bar graph showing IFN-Gamma PHA Stimulation.

FIG. 2D is a bar graph showing IL-4 PHA Stimulation.

FIG. 3A is a bar graph showing TCM induces IL-12 production in DC VIA TLR4.

FIG. 3B is a bar graph showing TCM induces IL-10 production in DC VIA TLR4.

FIG. 3C is a bar graph showing TCM induces CD80 in DC VIA TLR4.

FIG. 3D is a bar graph showing TCM induces CD86 in DC VIA TLR4.

FIG. 4 is a photograph of a two dimensional gel showing TCM 10%.

FIG. 5 is a photograph of a two dimensional gel showing TCM 16%.

FIG. 6 is a bar graph showing Imm-TCM augments PHA induced proliferation.

FIG. 7 is a bar graph showing Imm-TCM augments PHA induced IFN-Gamma.

FIG. 8 is a bar graph showing Imm-TCM decreases PHA induced IL-4.

FIG. 9 is a bar graph showing Imm-TCM increases NK Activity Against K562 (25:1).

### DESCRIPTION

Disclosed are compositions of matter derived from leukocyte lysate, dialysate, or lyophilized extracts that are capable of immune modulation. For the practice of the invention, the following terms are defined in relevance to generation and use of T cell modulator (TCM).

As used herein, the term “about” refers to a value that is within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values are provided in the application and claims, unless otherwise stated, the meaning of “about” or “comprising essentially of” should be assumed to be within an acceptable error range for that particular value.

“Specifically” binds, when referring to a ligand/receptor, antibody/antigen, or other binding pair, indicates a binding reaction which is determinative of the presence of the protein, e.g., TCM, in a heterogeneous population of proteins and/or other biologics. Thus, under designated conditions, a specified ligand/antigen binds to a particular receptor/antibody and does not bind in a significant amount to other proteins present in the sample.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell.

“Effective amount” encompasses an amount sufficient to ameliorate or prevent a symptom or sign of the medical condition. Effective amount also means an amount sufficient to allow or facilitate diagnosis. An effective amount for a particular subject may vary depending on factors such as the condition being treated, the overall health of the patient, the method route and dose of administration and the severity of side effects. An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

Dialysis.—The process of separating molecules in solution by the difference in their rates of diffusion through a semipermeable membrane. Leukocyte extract, after being performed separation and breakage of components, is placed in a semipermeable dialysis bag, such as a cellulose membrane with pores, and the bag is sealed. The sealed dialysis bag is placed in a container with a different solution, or pure water. Leukocyte extracts, being small enough to pass through the pores tend to move in or out of the dialysis bag in the direction of the lowest concentration. Larger molecules (often proteins, DNA, or polysaccharides) having significantly larger than the pore diameter are retained within the dialysis bag. In this way, separate leukocyte extracts less than or equal to 12,000 Daltons. f) filtration and sterilization.—After dialysis leukocyte extract is filtered through a membrane of pore size between 2 and 4 micrometers. Likewise, the solution sterilized again.

Formulation.—It takes a lyophilization process to remove leukocyte extract water through a vacuum generation, also in this stage involves the addition of a vehicle, such as milk, water, gel or flavoring artificial, to give a presentation and palatability of the product.

Physical Assessment. At this point we evaluated the physical and chemical processes such as density, pH, color, odor and taste. It is worth mentioning that if the product was not added any vehicle, transfer factor obtained is odorless, colorless and tasteless.

Assessment of biological activity. Leukocyte extract is analyzed by inoculating the extract in Balb-c at a concentration equivalent to that used in human leukocyte extract weight ratio, is performed by having kinetic inoculation mice exposed to extract for a specific time, for example 0, 2, 6, 24, 48 and 120 hours, blood is removed from mouse serum which served for the determination of activated cytosine placing the serum on microarrays membranes to determine the type of cytokine that is located in the leukocyte extract and uptime of the chemical signal in the induction of cytokines. Performing addition of serum dilutions to find the point where it no longer is cytosine, which means that the last dilution is the title of cytosine present. This means a degree or higher the dilution factor greater leukocyte extract power. Also, by study in time, ie kinetics, will be indicative of the starting time of the induction of cytokines, the optimal induction of cytokines and the total residence time of the induction of cytokines. Biological activity can be determined by use of T cell stimulation. In one embodiment Jurkat clone E6-1 cells (ATCC; Manassas, Va., USA is utilized). Briefly, 3×10⁵ cells are plated in 24-well plates and maintained in RPMI 1640 medium (ATCC; Virginia, USA) that is supplemented with fetal bovine serum (Gibco™, Life Technologies; New York, USA). Cultures are stimulated with leukocyte extract or TCM (0.1, 1, or 10μg/ml) or type IV-S concanavalin A (25 μg/ml; Sigma-Aldrich; Missouri, USA) as a positive control. After 72 h, IFN-γ was measured in the supernatant using the BDOptEIA-IFNγ ELISA kit (Becton Dickinson Biosciences; California, USA) by triplicate. After stimulation, 100 μl of standard or sample are mixed with 50 μl of PBS with 10% of heat inactivated FBS and incubated for two hours in capture antibody-coated wells. After extensive washing, 100 μl of a solution including a biotinylated detection antibody and streptavidin-horseradish peroxidase are added to each well and incubated for one hour. After washing, the reactions are developed with 3,3′5,5′-Tetramethylbenzidine (TMB) plus hydrogen peroxide, stopped, and read immediately at 450 nm. IFN-γ quantification above the detection limit (4.7 pg/ml) is considered as positive, since basal production of this cytokine in Jurkat cells is undetectable.

In one embodiment the invention is a composition containing the peptide sequence MXLLYAQDL/VEDN (SEQ ID NO. 1), or in another embodiment composition possesses an amino acid sequence with at least 20% homology to the following peptide sequence:

(SEQ ID NO. 2)
EFDVILKAAGANKVAVIKAVRGATGLGLKEAKDLVESAPAALKEGVSKDD
AEALKKALEEAGAEVEVK..

In one embodiment the composition possesses at least a 5 amino acid fragment that has an amino acid sequence with at least 20% homology to the following peptide sequence:

(SEQ ID NO. 2)
EFDVILKAAGANKVAVIKAVRGATGLGLKEAKDLVESAPAALKEGVSKDD
AEALKKALEEAGAEVEVK

The invention teaches treating a patient with an immunological disease comprising administration of a therapeutic amount of a leukocyte lysate extract. In said embodiment, leukocyte lysate extract is derived by the steps of: a) obtaining a population of leukocytes; b) lysing said leukocytes; c) extracting compounds of a molecular weight of approximately 12 KDa or less; and d) administering said extract into a patient in need of treatment. In one embodiment said leukocytes are extracted from a member of the chondrichthyans family. In one embodiment leukocyte extract is a composition that possesses an amino acid sequence with at least 20% homology to the following peptide sequence:

(SEQ ID NO. 2)
EFDVILKAAGANKVAVIKAVRGATGLGLKEAKDLVESAPAALKEGVSKDD
AEALKKALEEAGAEVEVK..

In one embodiment, said leukocyte extract possesses at least a 5 amino acid fragment that has an amino acid sequence with at least 20% homology to the following peptide sequence:

(SEQ ID NO. 2)
EFDVILKAAGANKVAVIKAVRGATGLGLKEAKDLVESAPAALKEGVSKDD
AEALKKALEEAGAEVEVK..

In another embodiment, A method of treating a cellular immune deficiency diseases in a diseased man is disclosed, said method comprising administering T cell modulator (TCM), a heat stable leucocyte extract obtained by drawing a blood sample, adding an EDTA type anticoagulant, separating white cells from the blood sample, suspending said white cells in saline solution and alternately freezing and thawing said suspension, thereafter lysing the suspension by incubation in the presence of magnesium and DNase, dialyzing the lysate against distilled water, separating the dialysate and lyophilizing the same, reconstituting the lyophilized product with distilled water, passing the product through a millipore filter and thereafter injecting said product.

In one embodiment of the invention dialyzable leukocyte extract from the antigenic polypeptides containing less than or equal to approximately 12,000 daltons, which is the spleen specific source that is part of the lymphatic system and is the center of activity selacimorfos immune system, which are the superorder chondrichthyans, commonly known as sharks, for obtaining transfer factor potentiated, which according to claim 1, characterized in that the product obtained is a transfer factor potentiated powdered form which can be easily transported and stored, also does not require refrigeration. Dialyzable leukocyte extract from the antigenic polypeptides containing less than or equal to approximately 12,000 daltons, which is the spleen specific source that is part of the lymphatic system and is the center of activity selacimorfos immune system, which are the superorder chondrichthyans, commonly known as sharks, for obtaining transfer factor potentiated, which according to claim 1, characterized in that it is obtained a power of transfer factor leukocytes 10¹²×mm³, defined as the concentration power leukocytes per ^{mm3} and the quality of the cells (smooth, round and inoquas). Dialyzable leukocyte extract from the antigenic polypeptides containing less than or equal to approximately 12,000 daltons, which is the spleen specific source that is part of the lymphatic system and is the center of activity selacimorfos immune system, which are the superorder chondrichthyans, commonly known as sharks, for obtaining transfer factor potentiated, which according to claim 1, characterized in that the method of testing leukocyte extract power from leukocyte extract inoculation in Balb-c, made in step of testing the biological activity consists of: Used groups of 8 mice, which were used at each time of the kinetics are inoculated with an amount equal to the weight-factor unit transfer (0.005 unit transfer factor) mice maintained at times appointed, with time 0 the basal level of cytokines induced mice, which were removed with the intention of determining the type, degree and permanence of the induced cytokines. Serum is removed from each mouse in time according to the kinetics and used 50 microliters of serum, exposed face of microarrays membranes containing receptor antibodies and cytokines wells to develop color will be induced cytokines. The serum was diluted with buffer solution in multiples of 2 dilutions initially and then in multiples of 100. It eliminates the time 0 baseline dilution and dilution to retain the color development in microarrays prior to dilution where no longer present the development of color, is the title of leukocyte extract. The group of mice that retain the title with the highest maximum induction time is the time spent by the induction of cytokines. Dialyzable leukocyte extract from the antigenic polypeptides containing less than or equal to approximately 12,000 daltons, which are the source or origin selacimorfos, commonly known with the name of sharks, or so-called shark, for obtaining transfer factor, characterized in that a greater degree and found induction time, higher power transfer factor. Dialyzable leukocyte extract from the antigenic polypeptides containing less than or equal to approximately 12,000 daltons, which is the spleen specific source that is part of the lymphatic system and is the center of activity selacimorfos immune system, which are the superorder chondrichthyans, commonly known as sharks, for obtaining transfer factor potentiated, which according to any preceding claim, characterized in that promotes cell excitation and optimization of chemical signals within the body of the individual who consumed. Dialyzable leukocyte extract from the antigenic polypeptides containing less than or equal to approximately 12,000 daltons, which is the spleen specific source that is part of the lymphatic system and is the center of activity selacimorfos immune system, which are the superorder chondrichthyans, commonly known as sharks, for obtaining transfer factor potentiated, which according to any preceding claim, characterized in that promotes a significantly increased activity of NK cells (natural murderer its acronym), the which provide virus protection as part of the innate immune defense system.

In one embodiment of the invention a “transfer factor” based procedure is utilized for generating TCM from donors that have not been immunized. In one specific embodiment shark splenocytes are used. Numerous descriptions of generating transfer factor are known in the literature. In practice of the invention, a preferred embodiment is the utilization of leukocytes from shark spleen that have been subjected to procedures useful for generation of transfer factor, these are well described in the following works, which are incorporated by reference U.S. Pat. Nos. 5,100,663, 4,616,079, 4,699,898, 4,710,380, 4,778,750, 4,874,608, 5,013,546, 5,081,108, 5,093,321.

In one embodiment buffy coat leukocytes, isolated from centrifugation of coagulated peripheral blood or splenocytes, is concentrated to 2×10(8) cells per ml in saline. The concentrated leukocytes or splenocytes are then subjected to 7 freeze-thaw cycles between −70 Celsius and 37 Celsius. Subsequent to freeze-thawing, the resultant substance is dialyzed for 24 hours utilizing an excess of sterile water over a peristaltic pump. The dialysate is then lyophilized in order to achieve concentration. Said concentrate is then ultrafiltered through a 10 kDa filter and heated to 60 Celsius. The material is subsequently filtered through a 2 micron filter, and lyophilized.

In another embodiment, shark splenocytes are centrifuged at 1000 rpm for 30 min at 4.degree. C, and resuspended in saline at 10(7) cells per ml and alternately frozen and thawed 10 times, using an acetone-dry ice mixture and a 37.degree. C water bath. Magnesium and DNase (Worthington Biochemical) are added, and the mixture is incubated at 37.degree. C for 30 min. The resultant cell lysate was dialyzed against 500 ml of distilled water in the cold for 2 days, and redialyzed by the same procedure. The dialysate (TCM) is lyophilized and stored at −20.degree. C until use, when it was dissolved in 2 ml of distilled water at room temperature and passed through a 0.45 .mu.m Millipore filter.

The resulting TCM, is utilized within the practice of the current invention for purposes of immune modulation. In one embodiment, TCM is administered sublingually for treatment of diseases associated with immune deregulation. In one embodiment TCM is administered for the treatment of vitiligo. Doses of administration are based on need required for immune modulation. In one embodiment a dose of approximately 1 mg of TCM protein is administered sublingually per day.

In one embodiment a composition is claimed, that is comprised of approximately 65% shark cartilage powdered at 200 mesh, 25% shark chondroitin, 5% probiotic blend, 5% turmeric curcuma longa extract. In said embodiment, probiotic blend is comprised of: Lactobacillus acidophilis, bifidobacterium bifidum, bifidobacterium longum, streptococcus thermophiles, lactobacillus bulgarcius, and lactobacillus paracasei.

In one embodiment, the invention teaches the use of TCM to augment immune responses to cancer, or as a direct anticancer agent. In one embodiment, TCM is utilized for stimulation of dendritic cells in vitro for use in ex vivo immunotherapy. TCM may be administered at various concentrations, in order to augment DC activation. Said DC activation may be measured by expression of costimulatory molecules, said costimulatory molecules are well known in the art and include CD80, CD86, CD40, ICOS, and OX2.

In another embodiment, TCM is administered as eyedrops for treatment of dry eye. In another embodiment an inflammatory condition is treated by administration of TCM. In another embodiment an autoimmune condition is treated by administration of TCM. The frequency of administration, as well as dosage are based on clinical and disease factors, these include stage of autoimmune disease, as well as patient specific factors. Factors of consideration include the amount of T cell autoreactivity that is ongoing as part of the autoimmune process. Specifically T cell autoreactivity may be assessed utilizing CD8 tetramers and flow cytometry, with said tetramers bearing autoantigen. Quantification of autoreactive T cell numbers may be performed by flow cytometry. Activation may be assessed by culture with said autoantigen and assessment of proliferation or cytokine production. Methods are known in the art for assessment of proliferation and autoantigen specific cytokine production such as thymidine incorporation and ELISPOT, respectively. Additional methods of assessing cytokine production include ELISA, Luminex, RT-PCR, Northern Blot and microarrays. Cytokines of interest include ones of specific relevance to autoimmunity including BLC, Eotaxin-1, Eotaxin-2, G-CSF, GM-CSF, 1-309, ICAM-1, IFN-gamma, IL-1 alpha, IL-1 beta, IL-1 ra, IL-2, IL-4, IL-5, IL-6, IL-6 sR, IL-7, IL-8, IL-10, IL-11, IL-12 p40, IL-12 p′70, IL-13, IL-15, IL-16, IL-17, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, P1GF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MT, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Angiostatin, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CAl25, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK.

Within the context of the invention is the sublingual use of TCM for stimulation of T regulatory cell function and/or number in a patient in need of immune modulation. A background on Treg cells will be provided to one of skill of the art a starting point for practice of the invention in light of stimulation of Treg for inhibition of autoimmunity. The concept of T cells suppressing other T cells as a mechanism of tolerance was accepted for decades. Initial studies in the 1970s focused on “T suppressor” cells, which were CD8 positive cells with the ability to restrain autoimmunity, support transplant tolerance, and were elevated in cancer. The existence of these cells came into doubt when molecular studies demonstrated fundamental proteins ascribed to these cells could not be found [1]. In the 1990s the focus started to shift to cells expressing the CD4+, CD25+ phenotype. The group of Hall et al were the first to describe a cell population with this phenotype capable of transferring tolerance in a rat model of transplantation [2, 3]. Subsequently, Sakaguchi's group, which are commonly given credit for identification of the Treg cell, confirmed the importance of the CD4+ CD25+ phenotype based on experiments demonstrating neonatal thymectomy causes loss of Treg, which results in systemic autoimmunity, which is prevented by transfer of the cell population [4]. Since those early days, the field of Treg has blossomed, with numerous molecular details of their function having been elucidated. Interestingly, observations made with the ill-defined T suppressor cells in the early 1980s, such as ability to suppress antigen presenting cell function [5], are now being rediscovered with Treg cells [6].

The invention comprises pharmaceutical formulations TCM. To prepare pharmaceutical or sterile compositions, the peptide, peptide mixture, or leukocyte lysate is admixed with a pharmaceutically acceptable carrier or excipient, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of TCM, administered alone or in combination with an immune modulatory agent, can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio between LD.sub.50 and ED.sub.50. The data obtained from these cell culture assays and animal studies can be used in formulating a range of dosage for use in humans. The dosage of such compounds lies preferably within a range of circulating concentrations that include the ED.sub.50 with little or no toxicity. The dosage may vary within this range depending upon the dosage form employed and the route of administration utilized.

In one embodiment of the invention, TCM is utilized in combination with agents possessing anticancer activity to augment efficacy in treatment of the tumor. Said agents are known in the art and include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Suitable routes of administration include parenteral administration, such as intramuscular, intravenous, or subcutaneous administration and oral administration. Administration of antibody used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection. In one embodiment, the binding compound of the invention is administered intravenously. In another embodiment, the binding compound of the invention is administered subcutaneously.

Alternately, one may administer TCM in a local rather than systemic manner, for example, via injection of the antibody directly into the site of action, often in a depot or sustained release formulation. Furthermore, one may administer the antibody in a targeted drug delivery system.

Guidance in selecting appropriate doses of biologics are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

TCM can be provided by continuous infusion, or by doses at intervals of, e.g., one day, one week, or 1-7 times per week. Doses may be provided intravenously, subcutaneously, intraperitoneally, cutaneously, topically, orally, nasally, rectally, intramuscular, intracerebrally, intraspinally, or by inhalation. A preferred dose protocol is one involving the maximal dose or dose frequency that avoids significant undesirable side effects. A total weekly dose is generally at least 0.05 .mu.g/kg body weight, more generally at least 0.2 .mu.g/kg, most generally at least 0.5 .mu.g/kg, typically at least 1 .mu.g/kg, more typically at least 10 .mu.g/kg, most typically at least 100 .mu.g/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg basis.

As used herein, “inhibit” or “treat” or “treatment” includes a postponement of development of the symptoms associated with disease and/or a reduction in the severity of such symptoms that will or are expected to develop with said disease. The terms further include ameliorating existing symptoms, preventing additional symptoms, and ameliorating or preventing the underlying causes of such symptoms. Thus, the terms denote that a beneficial result has been conferred on a vertebrate subject with a disease.

As used herein, the term “therapeutically effective amount” or “effective amount” refers to an amount of an TCM that when administered alone or in combination with an additional therapeutic agent to a cell, tissue, or subject is effective to prevent or ameliorate the disease or condition to be treated. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent are well known in the art, see, e.g., Hardman, et al. (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.

The pharmaceutical composition of the invention may also contain other agent, including but not limited to a cytotoxic, cytostatic, anti-angiogenic or antimetabolite agent, a tumor targeted agent, an immune stimulating or immune modulating agent or an antibody conjugated to a cytotoxic, cytostatic, or otherwise toxic agent. The pharmaceutical composition can also be employed with other therapeutic modalities such as surgery, chemotherapy and radiation.

Typical veterinary, experimental, or research subjects include monkeys, dogs, cats, rats, mice, rabbits, guinea pigs, horses, and humans.

### EXAMPLES

### Example 1

### Immune Modulation by TCM

Initial experiments assessed possible direct effects of TCM on cellular proliferation, given the reported antitumor activity, effects of TCM were assessed on HeLa cells, a cervical cancer cell line which has been utilized in cancer research for decades as an in vitro model of neoplasia. Originally derived in 1955, these cells are commonly utilized not for assessment of potentially useful anticancer agents, but also to assess non-specific inhibitory/cytotoxic activity of test compounds. Puck et al. Proc Natl Acad Sci USA. 1955 Jul. 15; 41(7):432-7 and Verma et al. Curr Med Chem. 2006; 13(4):423-48.

Production of the Th1 immunological cytokine interferon gamma (IFN-g) from human peripheral blood mononuclear cells (PBMC) was assessed directly by TCM, as well as the Th2 cytokine interleukin-4 (IL-4). In order to recapitulate in vivo effects assessment was performed of direct TCM stimulation of cytokine production, as well as addition of TCM to known cytokine inducer concanavalin A (ConA). IFNγ, is a cytokine that is critical for innate and adaptive immunity against viral and intracellular bacterial infections and for tumor control. IFNγ is an important activator of macrophages. Abnormal IFNγ expression is associated with a number of autoinflammatory and autoimmune diseases. The importance of IFNγ in the immune system stems in part from its ability to inhibit viral replication directly, and most importantly from its ability to stimulate and/or regulate the immune system. IFNγ is produced predominantly by natural killer (NK) and natural killer T (NKT) cells as part of the innate immune response, and by cytotoxic T lymphocyte (CTL) effector T cells¹². IL-4 is considered a prototypic Th2 cytokine, important in stimulation of antibody mediated immune responses, as well as generation of plasma cells. IL-4 is important in stimulating anti-inflammatory responses and has been used successfully in treatment of the mouse model of Type 1 diabetes, as well as other anti-inflammatory diseases. Accordingly, we assessed production of these two cytokines to gather an idea whether TCM acts on Th1 or Th2 cells, which is the broad classification of immune responses. ¹ Theofilopoulos A N. J Clin Invest. 2012 Oct. 1; 122(10):3464-6.² Roff et al. Front Immunol. 2014 Jan. 13; 4:49

T cell activation, and subsequent polarization into Th1 or Th2 subsets is controlled by mature dendritic cells which provide costimulatory molecules, in addition to antigen-MHC signals to the T cell. We sought to determine whether TCM induced maturation of dendritic cells, and assessed one of the major molecules involved in dendritic cell maturation, Toll like receptor (TLR)-4.

Given the reported dual roles of TCM in both immune stimulation (eg anticancer and antiviral) as well as immune regulation (efficacy in vitiligo and dry eye), and could be important for HIV patients and different cancer treatment by Dendritic Cells, we sought to determine whether TCM affected the generation of T regulatory cells.

### Materials and Methods

### Cell Lines

HeLa human cervical cancer cells were obtained from American Type Tissue Culture (ATCC: Manassas, Va.) and grown under fully humidified 5% CO2 environment with MEM supplemented with 10% FBS, 2% sodium pyruvate, non-essential amino acids (2 mM), penicillin (100 units/ml), streptomycin (100 μg/ml), and glutamine (4 mM) (Gibco-BRL). Cells were passaged by trypsinization twice weekly or as needed based on 75% confluency

### Peripheral Blood Mononuclear Cells (PBMC)

PBMC were isolated from buffy coats by density-gradient centrifugation. Specifically, buffy coat cells were dispensed over five 50 ml falcon tubes, phosphate-buffered saline (PBS)/2% fetal calf serum (FCS) solution was added to reach a volume of 20 ml and 10 ml Ficoll-Paque® was gently added under the diluted buffy coat cells. Centrifugation was performed at 400 g for 20 min at room temperature (RT) and washing of PBMC was done three times with PBS/2% FCS. Culture of freshly isolated PBMC was performed in complete MEM media.

### Cell Treatments and Analysis

TCM was diluted in complete MEM media prepared as described above. Dilutions of 1:10, 1:100, 1;1000 and 1:10,000 were performed. Negative controls were complete MEM media. Positive controls were concanavalin A at a concentration of 2.5 ug/ml. PBMC were plated at 1.5×10⁶ cells/ml in flat-bottom 96-well culture plates in a volume of 200 μl per well and incubated at 37° in a humidified 5% CO2 atmosphere. Conditioned media was then evaluated for IFN-gamma production using ELISA from R & D Systems (Quantikine ELISA). Concentration was calculated by plotting against a standard curve generated with control cytokine.

HeLa cells were plated at a concentration of 10,000 cells per well in flat bottom plates and incubated with dilutions of TCM at 1:10, 1:100, 1;1000 and 1:10,000. The 3-(4,5-dimethylthiazol-2-yl)-2,5-diphenyl tetrazolium bromide (MTT) assay was performed for assessment of proliferation. In this assay soluble MTT is metabolized by mitochondrial enzyme activity of viable tumor cells, into an insoluble colored formazan product. Subsequently formazan were dissolved in DMSO and measured spectrophotometrically at 540 nm. Briefly, 200 μl of cell suspension was seeded in 96-well microplates and incubated for 48 h (37° C., 5% CO2 air humidified).

To evaluate cell survival, 20 μl of MTT solution (5 mg/ml in PBS) was added to each well and incubated for 3 h. Then gently 150 μl of old medium containing MTT was replaced by DMSO and pipetted to dissolve any formed formazan crystals. Absorbance was then determined at 540 nm by enzyme-linked immunosorbent assay (ELISA) plate reader. Each extract concentration was assayed in 4 wells and repeated 3-times.

### ELISA

IFN-gamma, IL-4, IL-10 and IL-12 were assessed by ELISA (R and D Systems) utilizing supernatant from mitogen activated cultures and treated DC.

### Dendritic Cells

DC were generated from PBMC resuspended in RPMI-10% FCS, and allowed to adhere to 6-well plates (Costar Corp., Cambridge, Mass.). After 2 h incubation at 37 Celsius, the nonadherent cells were removed and the adherent cells washed in phosphate buffered saline (PBS), followed by detachment by incubation with Mg 2+ and Ca 2+ free PBS containing 0.5 mM EDTA at 37 Celsius. The adherent fraction was subsequently cultured at 3×10(6)/ml in RPMI-10% FCS supplemented with 50 ng/ml GM-CSF and 1,000 U/ml IL-4. Media is changed every 2 days for a total of 8 days culture. DC were isolated by positive selection for CD83 and subsequently treated with TCM on day 6 of culture. Assessment of maturation was performed by flow cytometry for CD80 and CD86 expression.

Blockade of TLR-4 was performed using by culture in the presence of TLR4 antagonist LPS-RS (Invivogen (San Diego, Calif.), (5μg/mL), with pretreatment 4 hours before exposure to TCM.

### Results

TCM does not Modulate Cellular Proliferation

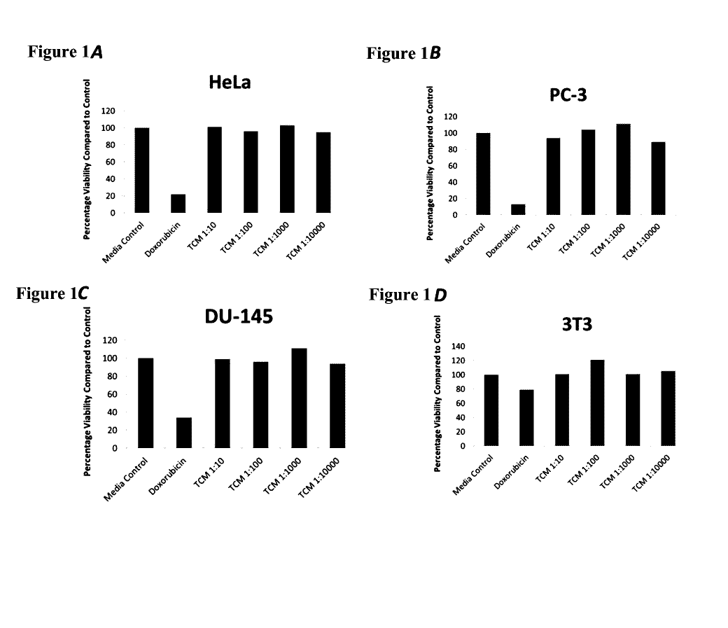

TCM has been reported to possess anticancer activity. Accordingly, we conducted a series of experiments assessing ability of various concentrations of TCM to inhibit proliferation of HeLa cells. We utilized the chemotherapeutic drug doxorubicin as a control. As seen in FIG. 1A, various doses of TCM did not affect proliferation of HeLa cells as assessed in the MTT assay after 48 hours of culture. Importantly, supraphysiological doses of TCM, as high as 1:10 diluted volume by volume in the tissue culture media did not result in inhibition of proliferation. These data suggest that TCM does not act through cytotoxic or cytostatic mechanisms. These data were confirmed with other cell lines such as PC-3, DU-145, and non-malignant 3T3 fibroblasts (FIGS. 1B-1D).

TCM Acts as a Cofactor for Cytokine Secretion from Immune Cells Found in Peripheral Blood

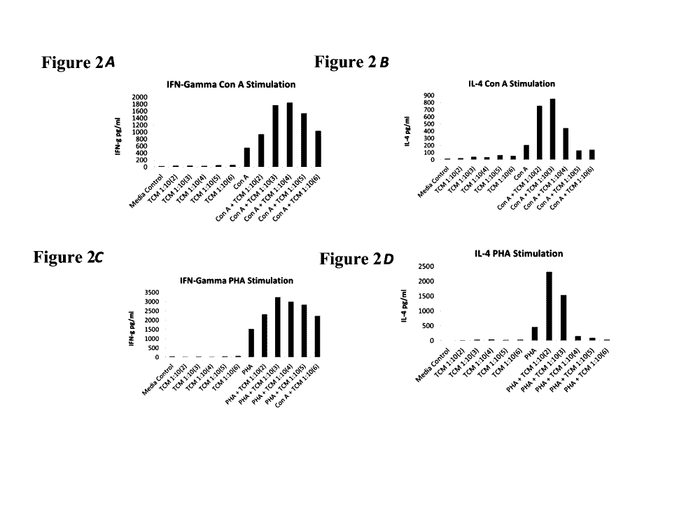

To assess whether TCM directly activates T cell production of cytokines, or whether it requires a costimulatory signal, such as concanavalin A (ConA), was examined. TCM did not affect viability of PBMC (data not shown). It appears that TCM complements existing production of immune stimulatory molecules after a primary stimuli, but does not initiate immunity, at least based on IFN-gamma and IL-4 production (FIGS. 2A and 2B). Given that different doses of TCM possess different costimulatory profiles for the different cytokines, we questioned whether the effect was specific to conconavalin A stimulation, or whether other factors may be at play. Accordingly, we substitute stimulation by conconavalin A to stimulation by phytohemagglutinin, a mitogen often used in studies stimulating human T cells. As seen in FIGS. 2C and 2D, a similar pattern of IFN-gamma and IL-4 costimulation was observed with PHA acting as the primary stimulator.

### TCM Induces Dendritic Cell Maturation in a TLR4 Dependent Manner

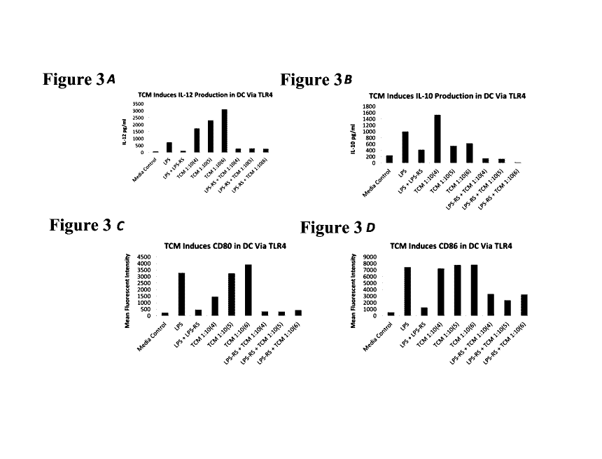

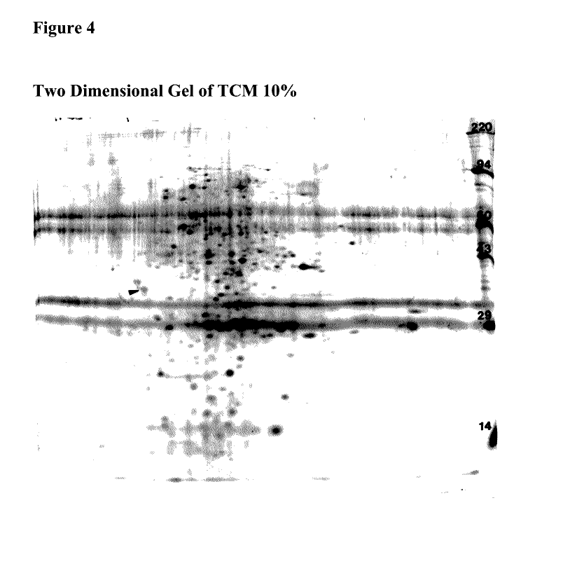

Given the contamination of antigen presenting cells in PBMC, and the fact that antigen presenting cells may be sending costimulatory signals to the T cells in response to TCM treatment, a series of experiments were conducted to assess whether TCM acts on the most potent antigen presenting cell, the dendritic cell. Day 6 immature DC generated from monocytes by IL4 and GM-CSF treatment were used to assess maturation-inducing potential of TCM. Cells were treated with saline, lps positive control, and 3 concentrations of TCM. Additionally, blockade of TLR4 signalling was performed by cotreatment with LPS-RS, an antagonist of the TLR-4 receptor. As seen in FIGS. 3A and 3B, TCM was capable of upregulating expression of IL-12 and IL-10, respectively, suggesting from a functional perspective that DC activation was occurring. Indeed the fact that IL-12 drives Th1 cytokine production and IL-10 drives Th2, these data are in agreement with the previous data suggesting that TCM is capable of modulating immunity. Definative evidence of maturation of DC was observed using flow cytometry, demonstrating that uprgulation of CD80 and CD86 was occurring as a result of TCM treatment (FIGS. 3C and 3D). In all experiments, blockade of TLR-4 by treatment with LPS-RS, an antagonist of TLR4, resulted in marked reduction of both LPS induced changes (positive control) as well as in activity of TCM.

### Example 2

### Molecular Characterization of TCM

### Two Dimensional Gel Electrophoresis

Two-dimensional electrophoresis was performed according to the carrier ampholine method of isoelectric focusing (O'Farrell, P. H., J. Biol. Chem. 250: 4007-4021, 1975, Burgess-Cassler, A., Johansen, J., Santek, D., Ide J., and Kendrick N., Clin. Chem. 35: 2297, 1989) by Kendrick Labs, Inc. (Madison, Wis.) as follows: Isoelectric focusing was carried out in a glass tube of inner diameter 2.3 mm using 2% pH 3-10 isodalt Servalytes (Serva, Heidelberg, Germany) for 9600 volt-hrs. One μg of an IEF internal standard, tropomyosin, was added to the sample. This protein migrates as a doublet with lower polypeptide spot of MW 33,000 and pI 5.2. The enclosed tube gel pH gradient plot for this set of Servalytes was determined with a surface pH electrode.

For the 10% acrylamide gels, after equilibration for 10 min in Buffer ‘0’ (10% glycerol, 50 mM dithiothreitol, 2.3% SDS and 0.0625 M tris, pH 6.8), each tube gel was sealed to the top of a stacking gel that overlaid a 10% acrylamide slab gel (0.75 mm thick). SDS slab gel electrophoresis was carried out for about 4 hrs at 15 mA/gel. The following proteins (Sigma Chemical Co., St. Louis, Mo. and EMD Millipore, Billerica, Mass.) were used as molecular weight standards: myosin (220,000), phosphorylase A (94,000), catalase (60,000), actin (43,000), carbonic anhydrase (29,000) and lysozyme (14,000). These standards appear along the basic edge of the silver-stained (Oakley, B. R., Kirsch, D. R. and Moris, N. R. Anal. Biochem. 105:361-363, 1980) 10% acrylamide slab gel. The gel was dried between sheets of cellophane with the acid edge to the left.

After equilibration for 15 min in Buffer “0” (10% glycerol, 50 mM dithiothreitol, 2.3% SDS and 0.0625 M tris, pH 6.8) each tube gel was sealed to the top of 10% acrylamide spacer gels which are on the top of 16.5% acrylamide peptide slab gels (Shagger, H. and Jagow, G. Anal. Biochem. 166: 368, 1987) (0.75 mm thick). SDS slab gel electrophoresis was started at 15 mamp/gel for the first four hours and then carried out overnight at 12 mamp/gel as for the separation of peptides. The slab gel electrophoresis was stopped after the bromophenol blue dye front had just started running off the gel. The following proteins (Sigma Chemical Co., St. Louis, Mo. and EMD Millipore, Billerica, Mass.) were added as molecular weight markers: phosphorylase A (94,000), catalase (60,000), actin (43,000) and lysozyme (14,000). These standards appear as bands on the basic edge of the silver stained (Oakley, B. R., Kirsch, D. R. and Moris, N. R. Anal. Biochem. 105:361-363, 1980) 16.5% acrylamide slab gel. Low molecular weight markers from Sigma Chemical were also loaded myoglobin (polypetide backbone) 1-153 16,950; Myoglobin (I+II, 1-131) 14,440; myoglobin (I+III, 56-153) 10,600; Myoglobin (I, 56-131) 8,160; myoglobin (II 1-55) 6,210; Glucagon 3,480; and Myoglobin (III, 132-153) 2,510. The gel was silver-stained and dried between sheets of cellophane paper with the acid edge to the left.

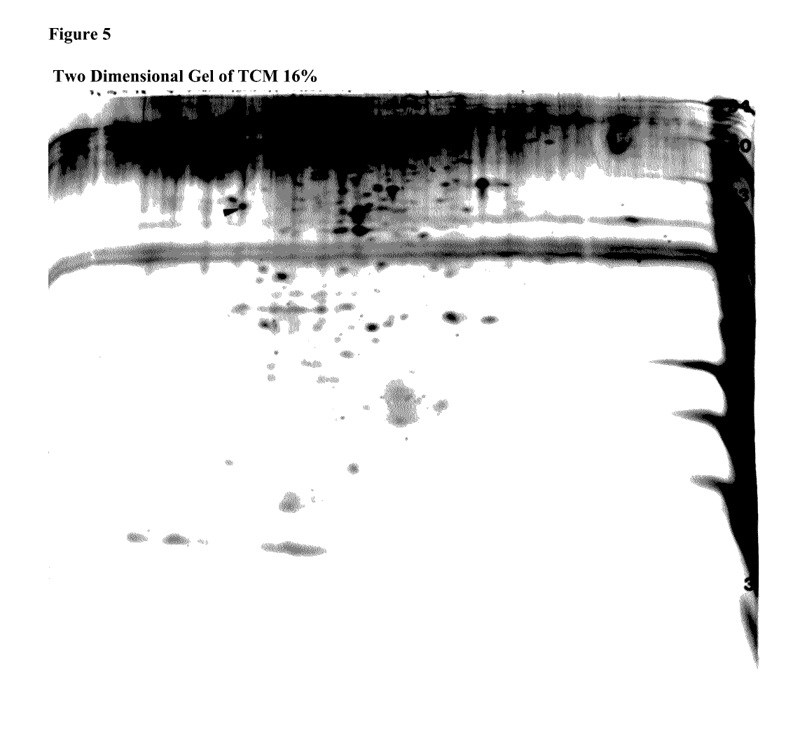

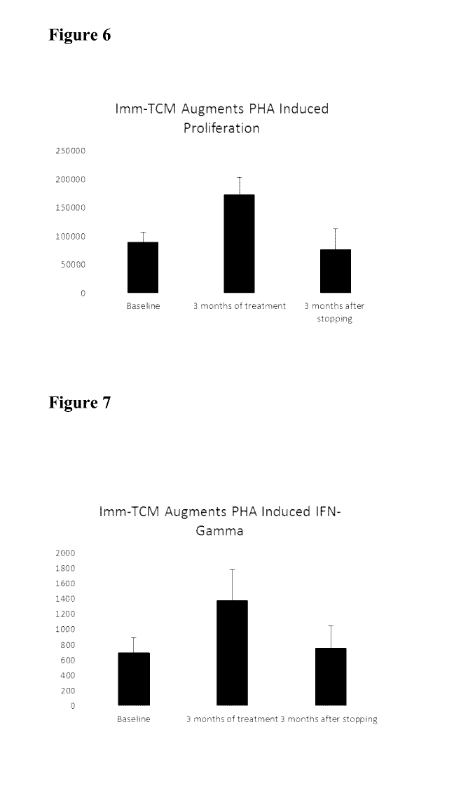

FIG. 5 illustrates the gel run under 10% conditions whereas FIG. 6 illustrates the gel run under 16% condition. The arrowhead illustrates the molecular weight spot indicative of the immune modulatory activity that was subsequently sequenced.

### Proteomic Analysis/Sequencing

Protein Digestion and Peptide Extraction.

Proteins that were separated by SDS-PAGE/2D-PAGE and stained by Coomassie dye were excised, washed and the proteins from the gel were treated according to published protocols [7-9]. Briefly, the gel pieces were washed in high purity, high performance liquid chromatography HPLC grade water, dehydrated and cut into small pieces and destained by incubating in 50 mM ammonium bicarbonate, 50 mM ammonium bicarbonate/50% acetonitrile, and 100% acetonitrile under moderate shaking, followed by drying in a speed-vac concentrator. The gel bands were then rehydrated with 50 mM ammonium bicarbonate. The procedure was repeated twice. The gel bands were then rehydrated in 50 mM ammonium bicarbonate containing 10 mM DTT and incubated at 56° C. for 45 minutes. The DTT solution was then replaced by 50 mM ammonium bicarbonate containing 100 mM Iodoacetamide for 45 minutes in the dark, with occasional vortexing. The gel pieces were then re-incubated in 50 mM ammonium bicarbonate/50% acetonitrile, and 100% acetonitrile under moderate shaking, followed by drying in speed-vac concentrator. The dry gel pieces were then rehydrated using 50 mM ammonium bicarbonate containing 10 ng/mL trypsin and incubated overnight at 37° C. under low shaking. The resulting peptides were extracted twice with 5% formic acid/50 mM ammonium bicarbonate/50% acetonitrile and once with 100% acetonitrile under moderate shaking. Peptide mixture was then dried in a speed-vac, solubilized in 20 □mL of 0.1% formic acid/2% acetonitrile.

LC-MS/MS.

The peptides mixture was analyzed by reversed phase liquid chromatography (LC) and MS (LC-MS/MS) using a NanoAcuity UPLC (Micromass/Waters, Milford, Mass.) coupled to a Q-TOF Ultima API MS (Micromass/Waters, Milford, Mass.), according to published procedures [7, 10-12]. Briefly, the peptides were loaded onto a 100 mm×10 mm NanoAquity BEH130 C18 1.7 mm UPLC column (Waters, Milford, Mass.) and eluted over a 150 minute gradient of 2-80% organic solvent (ACN containing 0.1% FA) at a flow rate of 400 nL/min. The aqueous solvent was 0.1% FA in HPLC water. The column was coupled to a Picotip Emitter Silicatip nano-electrospray needle (New Objective, Woburn, Mass.). MS data acquisition involved survey MS scans and automatic data dependent analysis (DDA) of the top three ions with the highest intensity ions with the charge of 2+, 3+ or 4+ ions. The MS/MS was triggered when the MS signal intensity exceeded 10 counts/second. In survey MS scans, the three most intense peaks were selected for collision-induced dissociation (CID) and fragmented until the total MS/MS ion counts reached 10,000 or for up to 6 seconds each. The entire procedure used was previously described [7, 10, 11]. Calibration was performed for both precursor and product ions using 1 pmol GluFib (Glu1-Fibrinopeptide B) standard peptide with the sequence EGVNDNEEGFFSAR (SEQ ID NO. 3) and the monoisotopic doubly-charged peak with m/z of 785.84.

Data Processing and Protein Identification.

The raw data were processed using ProteinLynx Global Server (PLGS, version 2.4) software as previously described [10]. The following parameters were used: background subtraction of polynomial order 5 adaptive with a threshold of 30%, two smoothings with a window of three channels in Savitzky-Golay mode and centroid calculation of top 80% of peaks based on a minimum peak width of 4 channels at half height. The resulting pkl files were submitted for database search and protein identification to the public Mascot database search (www.matrixscience.com), Matrix Science, London, UK) using the following parameters: databases from NCBI (all organisms, human proteins and rodent proteins for targeted identification of proteins), parent mass error of 1.3 Da, product ion error of 0.8 Da, enzyme used: trypsin, one missed cleavage, propionamide as cysteine fixed modification and Methionine oxidized as variable modification. To identify the false negative results, we used additional parameters such as different databases or organisms, a narrower error window for the parent mass error (1.2 and then 0.2 Da) and for the product ion error (0.6 Da), and up to two missed cleavage sites for trypsin. In addition, the pkl files were also searched against in-house PLGS database version 2.4 (www.waters.com) using searching parameters similar to the ones used for Mascot search. The Mascot and PLGS database search provided a list of proteins for each gel band. To eliminate false positive results, for the proteins identified by either one peptide or a mascot score lower than 25, we verified the MS/MS spectra that led to identification of a protein. The protein identified comprised of the amino acids:

(SEQ ID NO. 2)
EFDVILKAAGANKVAVIKAVRGATGLGLKEAKDLVESAPAALKEGVSKDD
AEALKKALEEAGAEVEVK.

### Example 3

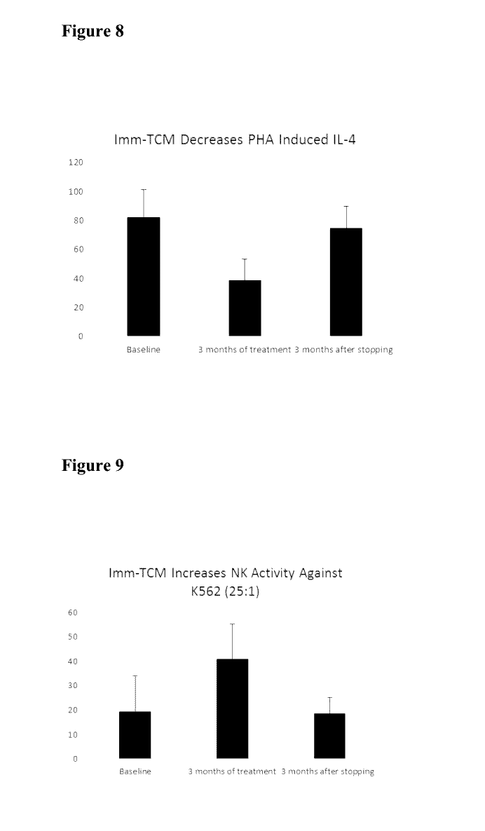

A composition of approximately 65% shark cartilage powdered at 200 mesh, 25% shark chondroitin, 5% probiotic blend, 5% turmeric curcuma longa extract. In said embodiment, probiotic blend is comprised of: Lactobacillus acidophilis, bifidobacterium bifidum, bifidobacterium longum, streptococcus thermophiles, lactobacillus bulgarcius, and lactobacillus paracasei, was administered daily in a volume of 200 mg per day twice a day for the period of 90 days. Blood markers and biochemistry where not significantly altered by IMM-TCM, when compared with baseline values. Additionally, immune modulation was noted as follows: a) augmentation of mitogen induced T cell proliferation (expressed as CPM) (FIG. 6); b) augmentation of T cell production of the immune stimulatory cytokine interferon gamma (expressed as pg/ml) (FIG. 7); c) suppression of T cell production of the Th2 cytokine interleukin-4 (expressed as pg/ml) (FIG. 8); and d) augmentation of natural killer cell activity (expressed as % lysis of K562 target cells) (FIG. 9). Interestingly, immune modulation was significantly lost after 90 days of cessation of IMM-TCM.

### REFERENCES

1. Germain, R. N., Special regulatory T-cell review: A rose by any other name: from suppressor T cells to Tregs, approbation to unbridled enthusiasm. Immunology, 2008. 123(1): p. 20-7.
2. Hall, B. M., et al., Specific unresponsiveness in rats with prolonged cardiac allograft survival after treatment with cyclosporine. III. Further characterization of the CD4+ suppressor cell and its mechanisms of action. J Exp Med, 1990. 171(1): p. 141-57.
3. Pearce, N. W., et al., Specific unresponsiveness in rats with prolonged cardiac allograft survival after treatment with cyclosporine. V. Dependence of CD4+ suppressor cells on the presence of alloantigen and cytokines, including interleukin 2. Transplantation, 1993. 55(2): p. 374-80.
4. Sakaguchi, S., et al., Immunologic self-tolerance maintained by activated T cells expressing IL-2 receptor alpha-chains (CD25). Breakdown of a single mechanism of self-tolerance causes various autoimmune diseases. J Immunol, 1995. 155(3): p. 1151-64.
5. Ptak, W. and R. K. Gershon, Immunological agnosis: a state that derives from T suppressor cell inhibition of antigen-presenting cells. Proc Natl Acad Sci USA, 1982. 79(8): p. 2645-8.
6. Min, W. P., et al., Inhibitory feedback loop between tolerogenic dendritic cells and regulatory T cells in transplant tolerance. J Immunol, 2003. 170(3): p. 1304-12.
7. Darie, C. C., et al., Identifying transient protein-protein interactions in EphB2 signaling by blue native PAGE and mass spectrometry. Proteomics, 2011. 11(23): p. 4514-28.
8. Shevchenko, A., et al., Mass spectrometric sequencing of proteins silver-stained polyacrylamide gels. Anal Chem, 1996. 68(5): p. 850-8.
9. Sokolowska, I., et al., Identification of potential tumor differentiation factor (TDF) receptor from steroid-responsive and steroid-resistant breast cancer cells. J Biol Chem, 2012. 287(3): p. 1719-33.
10. Sokolowska, I., et al., Proteomic analysis of plasma membranes isolated from undifferentiated and differentiated HepaRG cells. Proteome Sci, 2012. 10(1): p. 47.
11. Sokolowska, I., et al., Disulfide proteomics for identification of extracellular or secreted proteins. Electrophoresis, 2012. 33(16): p. 2527-36.
12. Spellman, D. S., et al., Stable isotopic labeling by amino acids in cultured primary neurons: application to brain-derived neurotrophic factor-dependent phosphotyrosine-associated signaling. Mol Cell Proteomics, 2008. 7(6): p. 1067-76.

What is claimed is:

1. A composition capable of modulating T cell immune responses, said composition extracted from immune cells by a dialysis method.

2. The composition of claim 1, wherein said composition is extracted from leukocytes.

3. The composition of claim 1, wherein said composition is extracted by homogenizing an immune organ.

4. The composition of claim 1, wherein said homogenizing of an immune organ is performed by mechanical means.

5. The composition of claim 1, wherein said homogenization of an immune organ is performed by ultasonication.

6. The composition of claim 1, wherein said immune organ is selected from a group of organs comprising of: a) spleen; b) lymph node; c) thymus; d) liver; e) peripheral blood mononuclear cells; f) Bursa of fabricius; and g) an organ containing a higher concentration of immune cells as compared to peripheral circulation.

7. The composition of claim 1, wherein said immune cells are obtained from a source that is autologous, allogeneic, or xenogeneic to the recipient.

8. The composition of claim 1, wherein said immune cells are derived from a member of the chondrichthyans family.

9. The composition of claim 1, wherein said immune cells are derived from a shark.

10. The composition of claim 2, wherein said leukocytes are broken down into proteins, lipids, and small molecules by use of a solvent.

11. The composition of claim 2, wherein said leukocytes are broken down into proteins, lipids, and small molecules by use of lyophilization.

12. The composition of claims 10 and 11 wherein said broken down leukocytes undergo an extraction process to isolate immune stimulatory fractions.

13. The composition of claim 12, wherein said extraction is performed by use of dialysis.

14. The composition of claim 13, wherein said dialysis is performed to extract molecules less than or equal to 12 kilodaltons.

15. The composition of claim 13, wherein non-denaturing conditions are used for extraction of a polypeptide substance capable of stimulating toll like receptor 4 (TLR4) on dendritic cells.

16. The composition of claim 15, wherein said stimulation of TLR4 on said dendritic cells is assessed by production of interleukin-12.

17. A composition that possesses an amino acid sequence with at least 20% homology to the following peptide sequence: EFDVILKAAGANKVAVIKAVRGATGLGLKEAKDLVESAPAALKEGVSKDDAEALKKALEEAGAE VEVK (SEQ ID NO: 2) that is capable of activating TLR-4.


SEQUENCE LISTING (3 sequences)

SEQ ID NO: 1 - protein, 12 aa, Artificial Sequence
  Description of Artificial Sequence Synthetic peptide
MXLLYAQDXEDN

SEQ ID NO: 2 - protein, 68 aa, Artificial Sequence [sequence omitted]

SEQ ID NO: 3 - protein, 14 aa, Artificial Sequence [sequence omitted]